(19) United States
(12) Patent Application Publication
(10) Pub. No.: US 2003/0087421 A1
(43) Pub. Date: May 8, 2003

(54) Lawsonia intracellularis

(76) Inventors: Connie J. Gebhart, Maple Grove, MN (US); Roberto Mauricio Carvalho Guedes, St. Paul, MN (US)

Correspondence Address: MARK S. ELLINGER, PH.D., Fish & Richardson P.C., P.A., Suite 3300, 60 South Sixth Street, Minneapolis, MN, 55402, US

(21) Appl. No.: 09/903,295

(22) Filed: Jul. 11, 2001

(51) Int. Cl. C12N 1/20

(52) U.S. Cl. 435/252.1; 435/252.3

(57) ABSTRACT

The invention provides methods and materials related to Lawsonia intracellularis microorganisms as well as methods for determining the efficacy of PE treatment materials. For example, the invention provides virulent L. intracellularis microorganisms as well as method for using virulent L. intracellularis microorganisms to test the efficacy of Lawsonia vaccines.

### BACKGROUND

1. Technical Field

The invention relates to Lawsonia intracellularis microorganisms as well as methods for determining the efficacy of proliferative enteropathy treatment materials such as Lawsonia vaccines.

2. Background Information

Proliferative enteropathy (PE) is a disease occurring worldwide in pigs. Both the subacute and chronic forms of PE are associated with reduced growth rate and diarrhea, observed most often in 6- to 20-week old pigs. The acute form of the disease consists of severe diarrhea and/or acute death in pigs 12 to 30 weeks old or older. The characteristic lesions consist of marked hyperplasia of enterocytes within crypts of the ileum, jejunum, cecum, or colon. A consistent feature is the presence of intracellular bacteria within the apical cytoplasm of proliferating crypt enterocytes. This intracellular bacterium has been identified as Lawsonia intracellularis, a gram-negative, obligately intracellular microorganism.

Avirulent L. intracellularis microorganisms such as those described in U.S. Pat. No. 5,885,823 have been used to make anti-L. intracellularis vaccines. Nevertheless, isolation and maintenance of L. intracellularis has been difficult.

### SUMMARY

The invention provides methods and materials related to L. intracellularis microorganisms. Specifically, the invention provides virulent L. intracellularis microorganisms, methods for isolating and maintaining virulent L. intracellularis microorganisms, and methods for determining the efficacy of PE treatment materials. PE treatment materials include, without limitation, any agent for treating, controlling, or preventing PE such as vaccines (e.g., Lawsonia vaccines), growth promoters, feed additives, pharmaceuticals, nutriceuticals, antibiotics, and antimicrobial agents. The vaccines can be attenuated live vaccines, modified live vaccines, recombinant vaccines, subunit vaccines, or nucleic acid vaccines.

L. intracellularis is a gram-negative, obligately intracellular microorganism. In nature, L. intracellularis can produce severe disease. In fact, natural infections of L. intracellularis can lead to death. Isolated L. intracellularis microorganisms can be either avirulent or virulent. Typically, isolated avirulent L. intracellularis microorganisms such as those described in U.S. Pat. No. 5,885,823 are used to make anti-Lawsonia intracellularis vaccines.

The invention is based on the discovery that existing L. intracellularis isolates do not induce Lawsonia infections to a degree suitable to assess the efficacy of PE treatment materials. For example, existing Lawsonia isolates, when administered to a pig, do not produce severe infections such as those infections resulting in gross intestinal lesions characteristic of PE. Thus, assessing PE treatment materials using existing Lawsonia isolates that do not produce severe Lawsonia infections can yield misleading results. The invention also is based on the discovery that virulent L. intracellularis microorganisms can be isolated and maintained such that severe Lawsonia infections can be induced in susceptible hosts. Such virulent L. intracellularis microorganisms can be used to test PE treatment materials. For example, animals vaccinated with a Lawsonia vaccine can be challenged with a virulent L. intracellularis microorganism provided herein to assess the effectiveness of the Lawsonia vaccine. In addition, the virulent L. intracellularis microorganisms provided herein can be used as positive controls in experiments designed to test the efficacy of PE treatment materials. Properly testing PE treatment materials using the materials and methods provided herein can be used identify and confirm the effectiveness of PE treatment materials that protect pigs worldwide from PE. The materials and methods provided herein also can be used to identify and confirm the effectiveness of PE treatment materials that prevent the transmission of L. intracellularis to other vertebrate species such as ferrets, foxes, poultry, horses, non-human primates, and humans.

In general, the invention features an isolated Lawsonia intracellularis microorganism, wherein administration of the microorganism to a pig produces at least one gross lesion (e.g., intestinal lesion). Administration of the microorganism to the pig can produce a gross lesion to the same degree as the gross lesion produced when the microorganism of ATCC accession number ______ (L. intracellularis isolate PHE/MN1-00) is administered to a susceptible pig. The microorganism can have the identifying characteristics of the microorganism of ATCC accession number (L. intracellularis isolate PHE/MN1-00). The microorganism can be obtained from the deposit having ATCC accession number (L. intracellularis isolate PHE/MN1-00). The microorganism could have been frozen. The microorganism could have been cultured for more than one passage. The microorganism could have been cultured for more than two passages. The microorganism could have been cultured for more than five passages. The microorganism could have been cultured for more than ten passages. The microorganism could have been cultured for less than twenty passages. The microorganism could have been cultured for less than ten passages. The microorganism could have been cultured for less than five passages. The microorganism can be in media. The microorganism can be in a eukaryotic cell (e.g., a McCoy, INT407, or IEC-18 cell).

In another aspect, the invention features a method for determining the efficacy of a PE treatment material, the method comprising (a) obtaining a vertebrate treated with the material, (b) infecting the vertebrate with a Lawsonia intracellularis microorganism, wherein administration of the Lawsonia intracellularis microorganism to a susceptible pig produces at least one gross lesion, and (c) determining the presence, absence, or severity of a Lawsonia infection in the vertebrate, wherein the degree of the Lawsonia infection inversely reflects the level of protection provided by the material. The material can be a vaccine (e.g., an attenuated live vaccine, modified live vaccine, recombinant vaccine, subunit vaccine, or nucleic acid vaccine). The material can be an antibiotic, a feed additive, or an antimicrobial agent. The material can lack live Lawsonia microorganisms. The infecting step can comprise intranasal administration, oral administration, intragastric administration, subcutaneous administration, or intramuscular administration. The vertebrate can be a pig, horse, hamster, mouse, rat, rabbit, or chicken. Administration of the microorganism to the susceptible pig can produce a gross lesion to the same degree as the gross lesion produced when the microorganism of ATCC accession number ______ (L. intracellularis isolate PHE/MN1-00) is administered to a different susceptible pig. The microorganism can have the identifying characteristics of the microorganism of ATCC accession number ______ (L. intracellularis isolate PHE/MN1-00). The microorganism can be obtained from the deposit having ATCC accession number (L. intracellularis isolate PHE/MN1-00).

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DETAILED DESCRIPTION

The invention provides methods and materials related to L. intracellularis microorganisms. Specifically, the invention provides virulent L. intracellularis microorganisms, methods for isolating and maintaining virulent L. intracellularis microorganisms, and methods for determining the efficacy of PE treatment materials.

The invention provides virulent L. intracellularis microorganisms. Typically, virulent L. intracellularis microorganisms, when administered to susceptible animals, produce severe Lawsonia infections. For example, administering the virulent L. intracellularis microorganisms provided herein to a susceptible animal can result in the formation of gross lesions in the intestine. The term “susceptible animal” as used herein with respect to administering virulent L. intracellularis microorganisms refers to an animal (e.g., vertebrate) that has not developed significant immunity against L. intracellularis. Examples of susceptible animals include those animals that have not been previously exposed to a L. intracellularis microorganism. The term “gross lesion” as used herein refers to an intestinal lesion that can be identified without the aid of a magnifying device. Typically, a gross lesion ranges in size from about 0.1 cm to about the entire length of an intestine. For example, a gross lesion can be about 0.5, 1, 2, 5, 10, 25, 50, 100, 150, 200, 250, 300, 350, 400, or more centimeters in length. Thus, a pig infected with a virulent L. intracellularis microorganism can have a gross lesion in its intestine that is 2 cm long or 25 cm long. In addition, animals infected with virulent L. intracellularis microorganisms can have multiple gross lesions in their intestines. In these cases, the size of each multiple gross lesion observed in the same intestine can be averaged for a representation of the overall gross lesion size for that intestine.

Gross lesions can be classified using a scoring system (e.g., a scale) that describes their appearance. For example, a gross lesion can be classified using a scale such that a value of 1 indicates mild mesenteric edema and hyperemia as well as mild intestinal wall edema and hyperemia; a value of 2 indicates mild to moderate edema and hyperemia of the mesentery and intestinal wall, and corrugated intestinal mucosa; a value of 3 indicates severe mesenteric edema and hyperemia as well as severe intestinal wall edema and hyperemia, and necrosis of the mucosal surface with formation of pseudo-diphtheric membrane; and a value of 4 indicates moderate to severe edema and hyperemia of the mesentery and intestinal wall, thick and corrugated mucosa, and blood clots in the intestinal lumen.

Virulent L. intracellularis microorganisms can be obtained from an infected vertebrate and maintained either extracellularly or intracellularly. When maintained extracellularly, the sample of virulent L. intracellularis microorganisms can be in a homogenate. The following techniques can be used to identify L. intracellularis-infected vertebrates and obtain virulent L. intracellularis microorganisms from those identified L. intracellularis-infected vertebrates.

Any method can be used to identify L. intracellularis-infected vertebrates. A Lawsonia infection can be identified by detecting the presence of L. intracellularis microorganisms, for example, by staining techniques, culturing techniques, PCR analysis, or by performing biochemical analyses such as IgA titration assays and delayed-type hypersensitivity assays. An L. intracellularis microorganism can be detected using L. intracellularis-specific antibodies. For example, a tissue sample from a pig suspected of having a Lawsonia infection can be tested using monoclonal antibodies that recognize L. intracellularis (McOrist et al., Vet. Rec., 121:421-422 (1987)). L. intracellularis microorganisms also can be detected using culturing techniques. For example, a sample from a pig suspected of having a Lawsonia infection can be used to challenge a culture of uninfected cells (e.g., McCoy cells). After a period of time (e.g. a week), the culture can be assessed by light microscopy or an immunofluorescence technique for the presence or absence of L. intracellularis microorganisms. Suitable samples for culture challenge include, without limitation, blood, intestine, and lymph node tissues.

In addition, L. intracellularis microorganisms can be detected by PCR analysis. For example, obtaining an amplification product specific for L. intracellularis chromosomal nucleic acid (e.g., a particular 328 bp L. intracellularis chromosomal gene sequence such as that disclosed in GenBank Accession No. L08049) can indicate the presence of an L. intracellularis microorganism. Examples of PCR primers designed to amplify such a chromosomal sequence include, without limitation, 5′-TATGGCTGTCAAACACTCCG-3′ (SEQ ID NO:1) and 5′-TGAAGGTATTGGTATTCTCC-3′ (SEQ ID NO:2). Suitable samples for PCR analysis include, without limitation, blood, feces, tissue, and mucosal samples (e.g., tonsil swabs, rectal swabs, and nasal swabs). PCR controls can include nucleic acid samples obtained from an animal know to have a L. intracellularis infection as well as nucleic acid samples obtained from an animal know to not have a L. intracellularis infection.

An IgA titration assay can be used to identify the presence of L. intracellularis. For example, an IgA titration assay can be used to determine the presence or absence of L. intracellularis-specific IgA present in a sample from a vertebrate. Briefly, a sample (e.g., an intestinal lavage sample) can be serially diluted, and each dilution can be added to a separate fixed culture of cells infected with virulent L. intracellularis microorganisms. After incubating (e.g., 30 minutes at 37° C.), the cultures can be washed and introduced to anti-IgA-specific antibodies conjugated with a label (e.g., peroxide). After another incubation, the cultures can be washed again, and the label can be developed and visualized. The presence of IgA antibodies in a sample from a vertebrate indicates the presence of L. intracellularis microorganisms in that vertebrate. Suitable samples for such an IgA titration assay include, without limitation, blood, serum, ascitic fluid, thoracic fluid, pericardial fluid, articular fluid, and cerebrospinal fluid.

Further, a delayed-type hypersensitivity (DTH) assay can be used to determine a vertebrate's DTH response to L. intracellularis antigens. Briefly, a vertebrate can be given intradermal injections of different amounts of an L. intracellularis antigen (e.g., formalin fixed L. intracellularis microorganisms, sonicated infected eukaryotic host cell suspensions, or sonicated L. intracellularis microorganisms). After a period of time (e.g., 24 or 48 hours), the vertebrate can be assessed for the presence or absence of a wheal and/or flare. The presence of a wheal and/or flare indicates that that vertebrate has been exposed to an L. intracellularis microorganism. The presence of a wheal and/or flare can be confirmed by comparing an L. intracellularis antigen test injection site to a control wheal and/or flare resulting from intradermally injecting a positive or negative control solution. Examples of a positive control solution include, without limitation, phytohemagglutinin (PHA). Examples of a negative control solution include, without limitation, sterile phosphate buffer saline (PBS) and McCoy cell suspensions.

Typically, a virulent L. intracellularis microorganism is isolated from a vertebrate having a severe Lawsonia infection. Any method can be used to identify a vertebrate having a severe Lawsonia infection. For example, the intestine of a pig suspected of having a severe Lawsonia infection can be examined for the presence of gross lesions. In addition, the degree of severity of a Lawsonia infection can be assessed using various methods. Such methods include, without limitation, observing clinical signs, performing quantitative serological assays, and examining tissue samples for lesions. For example, the clinical signs of a pig having a Lawsonia infection can be observed to assess the degree of severity of that Lawsonia infection. Clinical signs associated with a Lawsonia infection include, without limitation, fecal consistency, behavior, and body condition. A clinical sign can be classified using a scale that describes a range of observations related to that clinical sign. For example, fecal consistency can be classified using the following scale: 0=normal feces; 1=semi-solid feces with no blood; 2=watery stool with no blood; and 3=blood-tinged feces, loose or formed. Further, behavior and body condition can both be classified using the following scale: 0=normal; 1=slightly to moderately gaunt and depressed, listless, but still standing; and 2=severely gaunt, depressed, and recumbent. Classifying a vertebrate's clinical signs relates to the degree of severity of the Lawsonia infection in that vertebrate. For example, if a pig with a Lawsonia infection has loose or formed blood-tinged feces (i.e., a fecal consistency score of 3), is severely depressed and recumbent (i.e., a behavior score of 2), and is severely gaunt (i.e., a body condition score of 2), then the degree of severity of the Lawsonia infection in that pig is high. It is noted that avirulent strains of L. intracellularis such as those described in U.S. Pat. No. 5,885,823 produce Lawsonia infections with fecal consistency, behavior, and body condition scores of 0.

Quantitative serological assays can be used to assess the degree of severity of a Lawsonia infection. For example, serum samples from pigs that test positive for L. intracellularis-specific antibodies can be serially diluted (e.g., 1:30, 1:60, 1:90, 1:120, 1:240, and 1:480). Separate cultures of McCoy cells highly infected with L. intracellularis microorganisms can be incubated with an aliquot of each dilution. The infected cells can then be incubated to allow antibody interaction. The infected cells can then be washed, and L. intracellularis-specific antibodies can be detected using an isotype-specific antibody conjugated to a label (e.g., peroxidase). The labeled antibodies can be is developed, and the amount of L. intracellularis-specific antibodies can be evaluated by light microscopy. The amount of antibody in each dilution that recognizes L. intracellularis-specific antigens relates to the degree of severity of the Lawsonia infection in that vertebrate. For example, if cultured cells infected with L. intracellularis microorganisms are incubated with a serum sample of high dilution and that dilution tests positive for L. intracellularis-specific antibodies, then the vertebrate yielding that serum sample has a Lawsonia infection with a high degree of severity. Any sample that contains antibodies can be tested. Examples of such samples include, without limitation, blood, serum, ascitic fluid, thoracic fluid, and cerebrospinal fluid.

In addition to gross lesions, the severity of a Lawsonia infection can be assessed by examining tissue samples for the presence of histological lesions. The term “histological lesion” as used herein refers to those lesions that are not gross lesions. Any method can be used to identify a histological lesion, for example, immunohistochemical staining. Briefly, a sample from a vertebrate having a Lawsonia infection can be stained with antibodies that recognize L. intracellularis-specific antigens present in a histological lesion. Such antibodies have been described elsewhere (McOrist et al., Vet. Rec., 121:421-422, 1987). A histological lesion can be classified using a scale that describes the amount of L. intracellularis antigen associated with that histological lesion. For example, a histological lesion can be classified using the following scale: 0=no labeled antigen; 1=one isolated focal area of labeled antigen; 2=multifocal areas of labeled antigen; 3=majority of the mucosa has labeled antigen (e.g., between 40 and 80% of the mucosa); and 4=all of the mucosa has labeled antigen. The classification of histological lesions in a tissue sample from a vertebrate having a Lawsonia infection relates to the degree of severity of that Lawsonia infection. For example, a pig that has histological lesions wherein all of the surrounding mucosa has labeled antigen (i.e., a histological lesion score of 4) can be characterized as having a Lawsonia infection with a high degree of severity.

Once identified, a vertebrate having a Lawsonia infection such as a severe Lawsonia infection can be used to isolate virulent L. intracellularis microorganisms in the form of an intestinal homogenate. Briefly, a pig having a severe Lawsonia infection can be euthanized, and its ileum immediately removed. A section (e.g., about 2 cm long) can be cut from the ileum and immediately placed in a sterile container (e.g., a sterile petri dish). After briefly scraping the mucosal surface, about 2 mL of a sucrose-potassium-glutamate (SPG) solution containing 1% trypsin can be added to the petri dish. The section can be quickly homogenized (e.g., for 30 seconds). The resulting homogenate can be quickly and aseptically transferred to a 15 mL conical tube, and then agitated at 37° C. for a period of time (e.g., 35 minutes). The homogenate can be diluted with 10 mL SPG solution containing 10% fetal calf serum (FCS), and the resulting mixture further homogenized in a tissue grinder. The resulting homogenate can again be diluted with 10 mL SPG solution containing 10% FCS and then placed on ice. Debris can be removed from the mixture using sequentially more restrictive filters (e.g., sequentially through 2.7μ GF/D, 1.0μ, and 0.651μ filters). The final filtrated homogenate (˜10 mL) can be collected in a fresh sterile 15 mL conical tube and aliquoted aseptically into 1 mL cryovials. The aliquots of the homogenate can be placed on ice to be used for immediate infection or can be frozen immediately at −70° C. for future use.

In addition to maintaining virulent L. intracellularis microorganisms extracellularly in an intestinal homogenate, virulent L. intracellularis microorganisms can be maintained extracellularly in tissue culture media, blood, feces, tissue, or SPG buffer. While virulent L. intracellularis microorganisms can be maintained extracellularly, for example, in the form of homogenate, long term storage typically involves maintaining the virulent L. intracellularis microorganisms intracellularly since extracellularly maintained L. intracellularis microorganisms usually lose their virulence over time. Thus, the length of time maintained outside of a cell should be kept to a minimum if a highly virulent L. intracellularis microorganism is desired. For example, an L. intracellularis microorganism can be maintained extracellularly from about 1 minute to about 24 hours.

Virulent L. intracellularis microorganisms maintained extracellularly (e.g., virulent L. intracellularis microorganisms of an intestinal homogenate) can be used to prepare cells infected with virulent L. intracellularis microorganisms. Such infected cells can be used to maintain the virulent L. intracellularis microorganisms intracellularly. Any eukaryotic cell can be used to maintain a virulent L. intracellularis microorganism including, without limitation, McCoy cells, IEC-18 cells, and INT407 cells. Virulent L. intracellularis microorganisms can be introduced into eukaryotic cells using any method. For example, a fresh or previously frozen intestinal homogenate containing virulent L. intracellularis microorganisms can be added to a culture of host cells (e.g., 25 cm² tissue culture flask containing a culture of McCoy cells). The inoculated culture can be placed in a vacuum jar, and the atmosphere in the vacuum jar can be evacuated (e.g., to 500 mm Hg). After evacuating, the vacuum jar can be purged with hydrogen gas. Upon releasing the hydrogen gas pressure, the culture can be placed in an incubator under conditions that maintain L. intracellularis microorganism virulence (e.g., 8.8% CO₂, 8% O₂ and 83.2% N₂ at 37° C.). The inoculated culture can be refed after 3 to 6 hours with an appropriate growth and maintenance medium (e.g., DMEM containing 7% FCS, 0.5% neomycin, and 1.0% vancomycin). The inoculated cells can be passed frequently (e.g., once every week) to fresh cells until a desired number of cells are infected (e.g., 100% of the cells). Once infected, the cells can be stored frozen at, for example, −70° C.

Tissue culture cells infected with virulent L. intracellularis can be used to obtain a tissue culture homogenate containing virulent L. intracellularis microorganisms. For example, a tissue culture homogenate containing virulent L. intracellularis microorganisms can be prepared from L. intracellularis-infected McCoy cells, L. intracellularis-infected INT407 cells, or L. intracellularis-infected IEC-18 cells. Briefly, several cultures (e.g., twenty-seven 175 cm² flasks) of IEC-18 cells infected with virulent L. intracellularis microorganisms can be removed from their flasks, and the cell suspensions centrifuged to yield a cell pellet (e.g., 3,500× g for 20 minutes). The resulting cell pellet can be resuspended in a sucrose-potassium-glutamate (SPG) solution (e.g., a solution containing 0.218M sucrose, 0.0038M KH₂PO₄, 0.0072M K₂HPO₄, 0.0049M potassium glutamate, and 5% FBS). The SPG solution containing cells with virulent L. intracellularis microorganisms can be used to infect a vertebrate (e.g., a susceptible vertebrate to confirm virulence or a vaccinated vertebrate to test the vaccine's efficacy). Alternatively, the resulting cell pellet can be treated such that the cells are lysed, releasing the virulent L. intracellularis microorganisms. The released virulent L. intracellularis microorganisms can be filtered to remove cell debris, and the virulent L. intracellularis microorganisms used to infect a susceptible or vaccinated vertebrate.

Virulent L. intracellularis microorganisms (e.g., L. intracellularis isolate PHE/MN1-00) also can be obtained from The American Type Culture Collection (ATCC), 10801 University Boulevard, Manassas, Va. 20110-2209, USA (ATCC number ______, deposited on ______).

Various methods can be used to determine whether or not a particular L. intracellularis microorganism is virulent. For example, a susceptible vertebrate can be identified, infected with a sample containing the L. intracellularis microorganisms to be tested, and examined for the presence of a gross intestinal lesion.

Any method can be used to identify a vertebrate susceptible to a L. intracellularis infection. For example, serological tests can be used to confirm that a vertebrate (e.g., a pig) is negative for L. intracellularis. Serological tests also can be used to ensure that the vertebrate is not a host for other health-compromising microorganisms (e.g., porcine reproductive and respiratory syndrome virus (PRRSV), Actinobacillus pleuropneumonia, Salmonella cholerasuis, transmissible gastroenteritis virus (TGEV), Brachyspira pilosicoli, or Brachyspira hyodysenteriae). In addition, antibody screens can be used to confirm that a vertebrate lacks IgG antibodies specific for L. intracellularis. Further, fecal samples can be analyzed by, for example, PCR to confirm the lack of L. intracellularis nucleic acid (e.g., genomic DNA).

Once identified, the susceptible vertebrate can be infected with a sample of L. intracellularis microorganisms. Any method can be used to infect the susceptible vertebrate. For example, the susceptible vertebrate can be infected by intragastrically administering a sample of L. intracellularis microorganisms. Alternatively, the susceptible vertebrate can be infected by intranasally, orally, subcutaneously, or intramuscularly administering a sample of L. intracellularis microorganisms. Any sample of L. intracellularis microorganisms can be tested. For example, a tissue culture homogenate containing 8.0×10⁸ to 9.0×10⁸ L. intracellularis microorganisms per mL or an intestinal homogenate from a vertebrate suspected of having a Lawsonia infection and containing 2.5×10¹⁰ to 3.5×10¹⁰ microorganisms per mL can be used. Several days after infection (e.g., 20, 22, 24, 26, 28, or 30 days post infection), the vertebrate can be examined for the presence of a gross intestinal lesion. Briefly, the vertebrate can be euthanized, and its intestine removed. Once removed, the intestine can be visually examined for the presence or absence of gross lesions. The presence of a single visible gross lesion indicates that the L. intracellularis microorganism used to infect the vertebrate is virulent.

Positive and negative controls can be used when determining whether or not a particular L. intracellularis microorganism is virulent. For example, L. intracellularis isolate PHE/MN1-00 can be used as a positive control while the avirulent strains of L. intracellularis described in U.S. Pat. No. 5,885,823 can be used as negative controls.

Virulent L. intracellularis microorganisms can be propagated in a host. Hosts include, without limitation, McCoy cells, IEC-18 cells, INT407 cells, and vertebrates such as pigs, horses, chickens, turkeys, dogs, monkeys, and humans. For example, a culture of IEC-18 cells infected with virulent L. intracellularis microorganisms can be harvested (e.g., by cell scraping) and centrifuged to collect the infected cells. The collected cells can be resuspended in an appropriate growth and maintenance medium (e.g., 3 mL DMEM containing 5% FCS). A portion (e.g., 1 mL) of the resuspended infected cells can be added to an uninfected host. In another example, a vertebrate (e.g., a pig) can be inoculated with virulent L. intracellularis microorganisms or with cells containing virulent L. intracellularis microorganisms. In this case, the infected pig can develop a severe Lawsonia infection while serving as a host for virulent L. intracellularis microorganisms. When needed, the virulent L. intracellularis microorganisms can be isolated from the host pig using the methods provided herein. The isolated virulent L. intracellularis microorganisms can be passed on to another host for further propagation, or can be used to challenge a vertebrate when testing the efficacy of a PE treatment material.

Virulent L. intracellularis microorganisms can be used to determine the efficacy of PE treatment materials. Again, PE treatment materials include, without limitation, any agent for treating, controlling, or preventing PE such as vaccines (e.g., Lawsonia vaccines), growth promoters, feed additives, pharmaceuticals, nutriceuticals, antibiotics, and antimicrobial agents. Once given a particular PE treatment material, a vertebrate can be challenged with a virulent L. intracellularis microorganism. Once challenged, the vertebrate is assessed to determine the presence or absence of a Lawsonia infection. For example, a pig given a Lawsonia vaccine can be challenged with a virulent L. intracellularis microorganism provided herein and then assessed to determine the presence or absence of a Lawsonia infection. Determining the presence or absence of a Lawsonia infection in a treated vertebrate relates to the efficacy of that treatment material. For example, if a pig treated with a Lawsonia vaccine develops a Lawsonia infection after being challenged with a virulent L. intracellularis microorganism provided herein, then that Lawsonia vaccine is not effective in protecting that pig against a virulent L. intracellularis microorganism. Alternatively, if a pig treated with a Lawsonia vaccine does not develop a Lawsonia infection after being challenged with a virulent L. intracellularis microorganism provided herein, then that Lawsonia vaccine is effective in protecting that pig against a virulent L. intracellularis microorganism.

In addition, the severity of a Lawsonia infection can be used to assess the efficacy of a PE treatment material. For example, a pig given a particular PE treatment material can be challenged with a virulent L. intracellularis microorganism and then assessed to determine the severity of any resulting Lawsonia infection. If the treated pig challenged with a virulent L. intracellularis microorganism develops a Lawsonia infection to a lesser extent than control pigs, then the degree of efficacy for that particular treatment can be determined.

The invention will be further described in the following examples, which do not limit the scope of the invention described in the claims.

### EXAMPLES

### Example 1

### Analyzing Lawsonia intracellularis Infections in Pigs

Twenty-four 4 to 5-week-old pigs weighing between 20 and 30 pounds were obtained from a herd with no history or recorded cases of PE. The herd was confirmed serologically negative for L. intracellularis, porcine reproductive and respiratory syndrome virus (PRRSV), Actinobacillus pleuropneumonia, Salmonella cholerasuis, transmissible gastroenteritis virus (TGEV), Brachyspira pilosicoli and Brachyspira hyodysenteriae. Forty-eight hours before challenging, blood and fecal samples were collected from each of the 24 pigs. The blood samples were analyzed serologically using the immunoperoxidase monolayer assay (IPMA) according to the method of Guedes et al. (Guedes et al., Internat. Pig Vet. Soc. Congress, p.81, 2000) to confirm that the pigs were negative for IgG antibodies specific for L. intracellularis microorganisms. The fecal samples were analyzed by PCR to confirm the absence of L. intracellularis microorganisms, ensuring PE negativity. Twenty-four hours before challenging, the pigs were divided randomly by weight into three groups: a control group containing 4 pigs, a tissue culture homogenate group containing 10 pigs, and an intestine homogenate group containing 10 pigs. Each group was housed in a separate room in an isolation barn.

The challenges were administered on day 0 to each pig intragastrically using a stomach tube. The pigs in the control group were challenged with 40 mL of sucrose-potassium-glutamate (SPG) solution. The pigs in the tissue culture homogenate group were challenged with 40 mL SPG solution containing 8.9×10⁸ L. intracellularis microorganisms from a tissue culture homogenate. The pigs in the intestine homogenate group were challenged with 40 mL SPG solution containing 2.9×10¹⁰ L. intracellularis microorganisms scraped from the necrospied intestinal mucosa of a young female pig infected with an acute form of PE. Both inocula were quantitated by immunoperoxidase using a monoclonal antibody specific for L. intracellularis microorganisms. Briefly, 15-well glass slides were coated with 10 μL of serial 1:10 dilutions of each inoculum. The coated slides were dried for 30 minutes at 37° C. The inocula dilutions dried on the slides were fixed with cold acetone for 20 minutes at 37° C. and then stained using a monoclonal antibody specific for L. intracellularis, described elsewhere (McOrist et al., Vet. Rec.,121:421-422, 1987). L. intracellularis microorganisms were visualized using peroxidase-conjugated streptavidin (LSAB2-K0675-11; DAKO USA, Carpinteria, Calif.), and the number of microorganisms was counted using a light microscope.

Clinical observations including fecal consistency, behavior, and body condition were made on a daily basis from day 1 until day 22. Fecal consistency was scored based on the following scale: 0=normal feces; 1=semi-solid feces with no blood; 2=watery without dark or bloody feces; and 3=blood-tinged feces, loose or formed. Behavior and body condition were scored based on the following scale: 0=normal; 1=slightly to moderately gaunt and depressed, listless, but still standing; and 2=severely gaunt and depressed, recumbent. Fecal samples collected 3 times per week were analyzed for the presence of L. intracellularis microorganisms by PCR according to a method described elsewhere (Jones et al., J. Clinic. Microbiol., 31:2611-2615, 1993). Blood samples collected on days 0, 7, 14, and 20 were analyzed using the ELISPOT T cell assay described in Example 2 and by IPMA (Guedes et al, 2000).

On day 21, each pig was sedated, and 200 μL of ten different solutions were injected intradermally in ten different areas between the nipples. The ten different solutions were as follows: (1) sterile phosphate buffer saline (PBS) as a negative control; (2) 33 μg sonicated non-infected McCoy cells suspension as a negative control; (3) 50 μg of phytohemagglutinin (PHA) as a positive control; (4) 10⁹ formalin fixed L. intracellularis microorganisms per mL; (5) 10⁸ formalin fixed L. intracellularis microorganisms per mL; (6) 10⁷ formalin fixed L. intracellularis microorganisms per mL; (7) 50 μg sonicated L. intracellularis microorganisms; (8) 5 μg sonicated L. intracellularis microorganisms; (9) 15 μg isolated outer membrane protein from L. intracellularis microorganisms; and (10) 1.5 μg isolated outer membrane protein from L. intracellularis microorganisms. Delayed type hypersensitivity immune response was measured using a manual caliper 24 and 48 hours after the injections.

Each pig was weighed and euthanized 22 days after inoculation. Intestinal lavage of the aboral 25 cm of the small intestine of each pig was performed using 20 mL cold PBS. Each 20 mL intestinal lavage sample was then centrifuged at 800 rpm for 5 minutes to eliminate solid material. The supernatant from each centrifuged sample was frozen and analyzed later using the IgA titration assay described in Example 3.

Gross lesions in jejunum, ileum, cecum, and colon samples were evaluated and scored separately in each pig using the following scale: 0=no gross lesions; 1=mild mesenteric edema and hyperemia as well as mild intestinal wall edema and hyperemia; 2=mild to moderate edema and hyperemia of the mesentery and intestinal wall, and corrugated intestinal mucosa; 3=severe mesenteric edema and hyperemia as well as severe intestinal wall edema and hyperemia, and necrosis of the mucosal surface with formation of pseudo-diphtheric membrane (necrotic enteritis); and 4=moderate to severe edema and hyperemia of the mesentery and intestinal wall, thick and corrugated mucosa, and blood clots in the intestinal lumen. The length of each gross lesion within each intestinal section was measured and recorded. After evaluating the samples for gross lesions, the ileum samples were fixed by immersion in 10% neutral buffered formalin. The formalin fixed ileum samples were embedded in paraffin and cut into 5 μm thick sections. A section from each sample was stained by hematoxylin and eosin, and was evaluated for histological lesions. Another section from each sample was immunostained with mouse monoclonal antibodies recognizing L. intracellularis. L. intracellularis microorganisms were visualized using peroxidase-conjugated streptavidin (LSAB2-K0675-11; DAKO USA). Histological lesions were evaluated by fluorescence microscopy and were scored using the following scale: 0=no labeled antigen; 1=one isolated focal area of labeled antigen; 2=multifocal areas of labeled antigen; 3=majority of the mucosa has labeled antigen (e.g., between 40 and 80% of the mucosa); and 4=all of the mucosa has labeled antigen.

Pigs in the control group exhibited fecal consistency scores of 0 and 1 from −2 to 21 days post-inoculation, with the majority of pigs exhibiting scores of 0. The majority of pigs in both the tissue culture homogenate and intestine homogenate groups at 2 days prior to inoculation (i.e., −2 days post-inoculation) exhibited fecal consistency scores of 0 and 1. At day 7 post-inoculation, seven, two, and one pig(s) in the tissue culture homogenate group exhibited scores of 0, 1, and 2, respectively, while eight, one, and one pig(s) in the intestine homogenate group exhibited scores of 0, 1, and 3, respectively. The majority of pigs in both the tissue culture homogenate and intestine homogenate groups exhibited scores of 1 or greater from day 14 to day 21 post-inoculation (Table 1).

TABLE 1
Fecal scoring of pigs from days −2, 7, and 14-21 post-inoculation.

| | | Day | | | | | | | | | |
|---|---|---|---|---|---|---|---|---|---|---|---|
| Groups | Fecal score* | −2 | 7 | 14 | 15 | 16 | 17 | 18 | 19 | 20 | 21 |
| Control (n = 4) | 0 | 4 | 2 | 3 | 2 | 3 | 4 | 3 | 4 | 3 | 2 |
| | 1 | 0 | 2 | 1 | 2 | 1 | 0 | 1 | 0 | 1 | 2 |
| | 2 | 0 | 0 | 0 | 0 | 0 | 0 | 0 | 0 | 0 | 0 |
| | 3 | 0 | 0 | 0 | 0 | 0 | 0 | 0 | 0 | 0 | 0 |
| Tissue culture homogenate | 0 | 9 | 7 | 4 | 4 | 5 | 5 | 5 | 4 | 4 | 4 |
| (n = 10) | 1 | 1 | 2 | 6 | 6 | 5 | 5 | 5 | 6 | 6 | 6 |
| | 2 | 0 | 1 | 0 | 0 | 0 | 0 | 0 | 0 | 0 | 0 |
| | 3 | 0 | 0 | 0 | 0 | 0 | 0 | 0 | 0 | 0 | 0 |
| Intestine homogenate (n = 10) | 0 | 0 | 8 | 5 | 3 | 3 | 5 | 4 | 3 | 2 | 3 |
| | 1 | 0 | 1 | 5 | 6 | 6 | 4 | 5 | 6 | 7 | 6 |
| | 2 | 0 | 0 | 0 | 1 | 1 | 0 | 1 | 1 | 1 | 1 |
| | 3 | 0 | 1 | 0 | 0 | 0 | 1 | 0 | 0 | 0 | 0 |

*0 = normal feces; 1 = semisolid feces, with no blood; 2 = watery stool, runs through the floor slats, with no blood; and 3 = blood tinged feces, loose or formed.

All pigs in all groups displayed a negative delayed-type hypersensitivity (DTH) response to the negative controls (PBS and McCoy cells suspension). In addition, all pigs in all groups exhibited a positive DTH response to the positive control (phytohemagglutinin). The pigs in both the tissue culture homogenate and intestine homogenate groups exhibited dose-dependent DTH responses to L. intracellularis whole bacteria, L. intracellularis whole sonicated bacteria, as well as purified L. intracellularis outer membrane proteins compared to the control group pigs as assessed by erythema (i.e., flare or redness). All pigs showed no substantial difference in injection site thickness (i.e., wheal or unduration) (Table 2).

TABLE 2
Delayed-type hypersensitivity (DTH) response in pigs 24 and 48 hours after
injections done on day 20 post-inoculation.

| End | | | | | | | | | | | | |
|---|---|---|---|---|---|---|---|---|---|---|---|---|
| points | Hrs | Groups | A* | B | C | D | E | F | G | H | I | J |
| Erythema | 24 | Control (4) | 0 | 0 | 16.4 | 4.9 | 0 | 0 | 3.4 | 0 | 2.4 | 0 |
| (mm) | | Tissue culture | 0 | 0 | 13.25 | 6.9 | 2.58 | 0.67 | 1.5 | 0.64 | 0.63 | 0 |
| | | homogenate (10) | | | | | | | | | | |
| | | Intestine | 0 | 0 | 11.46 | 8.07 | 3.39 | 0.5 | 3.76 | 1.12 | 1.64 | 0.39 |
| | | homogenate (10) | | | | | | | | | | |
| | 48 | Control(4) | 0 | 0 | 10.5 | 1.5 | 0 | 0 | 0 | 0 | 0 | 0 |
| | | Tissue culture | 0 | 0 | 8.85 | 5.06 | 0.06 | 0 | 0 | 0 | 0 | 0 |
| | | homogenate (10) | | | | | | | | | | |
| | | Intestine | 0 | 0 | 7.67 | 6.14 | 1.67 | 0 | 0.95 | 0.58 | 0 | 0 |
| | | homogenate (10) | | | | | | | | | | |
| Thickness | 24 | Control (4) | 3 | 3 | 6.8 | 3 | 3 | 3 | 3 | 3 | 3 | 3 |
| (mm) | | Tissue culture | 3 | 3 | 8.45 | 5.39 | 3 | 3 | 3 | 3 | 3 | 3 |
| | | homogenate (10) | | | | | | | | | | |
| | | Intestine | 3 | 3 | 7.41 | 5.34 | 3 | 3 | 3 | 3 | 3 | 3 |
| | | homogenate (10) | | | | | | | | | | |
| | 48 | Control (4) | 3 | 3 | 3.6 | 3 | 3 | 3 | 3 | 3 | 3 | 3 |
| | | Tissue culture | 3 | 3 | 6.06 | 3.42 | 3 | 3 | 3 | 3 | 3 | 3 |
| | | homogenate (10) | | | | | | | | | | |
| | | Intestine | 3 | 3 | 4.94 | 4.68 | 3 | 3 | 3 | 3 | 3 | 3 |
| | | homogenate (10) | | | | | | | | | | |
| *Antigens used: A = PBS; B = McCoy cells suspension; C = PITA; D = L. intracellularis whole bacteria, 10⁹ organisms per ml; E = L. intracellularis whole bacteria, 10⁸ organisms per ml; F = L. intracellularis whole bacteria, 10⁷ organisms per ml; G = L. intracellularis whole bacteria sonicated, 250 μg per ml; H = L. intracellularis whole bacteria sonicated, 25 μg per ml; I = L. | | | | | | | | | | | | |
| #intracellularis outer-membrane proteins, 75 μg per ml; and J = L. intracellularis outer-membrane proteins, 7.5 μg per ml. | | | | | | | | | | | | |

All four of the pigs in the control group lacked gross lesions (e.g., a score of 0). Two pigs from the tissue culture homogenate group exhibited gross lesion scores of 1, while the remaining eight pigs exhibited gross lesion scores of 2. Two pigs from the intestine homogenate group exhibited gross lesion scores of 1, while five pigs and three pigs exhibited gross lesion scores of 2 and 3, respectively. The average gross lesion length for the tissue culture homogenate group was 21.2 cm, and the average gross lesions length for the intestine homogenate group was 46.1 cm (Table 3).

TABLE 3
Gross lesion score and average of gross lesion length (cm) in pigs on
day 22 post-inoculation.

| | Gross lesion score | | | | | |
|---|---|---|---|---|---|---|
| Groups | 0* | 1 | 2 | 3 | 4 | Average length** |
| Control (n = 4) | 4 | 0 | 0 | 0 | 0 | 0 |
| Tissue culture homogenate | 0 | 2 | 8 | 0 | 0 | 21.2 |
| (n = 10) | | | | | | |
| Intestine homogenate (n = 10) | 0 | 2 | 5 | 3 | 0 | 46.1 |
| *0 = no gross lesions; 1 = mild mesenteric edema and hyperemia as well as mild intestinal wall edema and hyperemia; 2 = mild to moderate edema and hyperemia of the mesentery and intestinal wall, and corrugated intestinal mucosa; 3 = severe mesenteric edema and hyperemia as well as severe intestinal wall edema and hyperemia, and necrosis of the mucosal surface with formation of pseudo-diphtheric membrane; 4 = moderate to severe edema and hyperemia of the | | | | | | |
| #mesentery and intestinal wall, thick and | | | | | | |

corrugated mucosa, and blood clots in the intestinal lumen. ** Average length of lesions within group.

All pigs in the control group were negative for immunoreactive histological lesion in any of the tissues analyzed. Pigs in both the tissue culture homogenate group and the intestine homogenate group exhibited histological lesion scores of 2 or greater in ileum sections. The histological lesions in pigs from both groups were less severe (i.e., had lower scores) in jejunum and colon/cecum sections. The majority of pigs in both groups also exhibited Lawsonia antigen by immunohistochemistry in ileo-cecal lymph node sections (Table 4).

TABLE 4
Histological lesion score in pigs on day 22 post-inoculation.

| | | | | colon/ | Ileo-cecal |
|---|---|---|---|---|---|
| Groups | Score* | Ileum | Jejunum | cecum | lymph node** |
| Control (n = 4) | 0 | 4 | 4 | 4 | 4 |
| | 1 | 0 | 0 | 0 | 0 |
| | 2 | 0 | 0 | 0 | — |
| | 3 | 0 | 0 | 0 | — |
| | 4 | 0 | 0 | 0 | — |
| Tissue culture | 0 | 0 | 5 | 3 | 2 |
| homogenate (n = 10) | | | | | |
| | 1 | 0 | 3 | 5 | 7 |
| | 2 | 5 | 1 | 2 | — |
| | 3 | 3 | 1 | 0 | — |
| | 4 | 2 | 0 | 0 | — |
| Gut homogenate (n = 10) | 0 | 0 | 4 | 1 | 0 |
| | 1 | 0 | 2 | 4 | 9 |
| | 2 | 3 | 0 | 4 | — |
| | 3 | 2 | 2 | 1 | — |
| | 4 | 5 | 1 | 0 | — |

*0 = no labeled antigen; 1 = one isolated focal area of labeled antigen; 2 = multifocal areas of labeled antigen; 3 = majority of the mucosa has labeled antigen; and 4 = all of the mucosa has labeled antigen.
**Presence (1) or absence (0) of labeled antigen in lymph node sections

These data demonstrated that clinical signs (e.g., fecal consistency) and the characterization of gross and histological lesions can be used to assess the severity of a Lawsonia infection. Further, these data demonstrated that DTH response can be used to determine whether a vertebrate has been exposed to a Lawsonia infection. In addition, these data demonstrated that a majority of pigs infected with a virulent L. intracellularis microorganism exhibit (1) fecal consistency scores of 1 or greater, (2) dose-dependent DTH responses, (3) gross lesion scores of 2 or greater, and (4) ileal histological lesion scores of 2 or greater.

### Example 2

### IPMA Serological Assay

Serum samples from pigs were diluted 1:30 in PBS. Aliquots (50 μL) of each diluted serum sample were added to separate test wells in a 96-well plate containing acetone-fixed McCoy cells infected with L. intracellularis. After incubating at 37° C. for 30 minutes, the wells were washed five times with PBS. Following washing, 30 μL aliquots of anti-porcine IgG-peroxidase conjugate (1:600; A-7402; Sigma) were added to each well. After incubating at 37° C. for 45 minutes, the wells were washed five times with PBS. The peroxidase-conjugated antibodies were developed by adding 100 μL prediluted chromogen solution (AEC: 3-amino-9-ethyl-carbazole) to each well. After incubating at room temperature for 20 minutes, the wells were washed three times with PBS, allowed to dry, and examined using an inverted microscope.

All four pigs in the control group tested negative for IgG antibodies against L. intracellularis from 2 days prior to inoculation (i.e., −2 days post-inoculation) to 20 days post-inoculation. At days −2 and 7 post-inoculation, all pigs in both the tissue culture homogenate and intestine homogenate groups were negative for IgG antibodies against L. intracellularis. At day 14 post-inoculation, one pig, three pigs, and one pig in the tissue culture homogenate group exhibited IgG antibodies against L. intracellularis with titers of 1:60, 1:120, and 1:240, respectively, while one pig and two pigs in the intestine homogenate group exhibited IgG antibodies against L. intracellularis with titers of 1:30 and 1:60, respectively. By day 20 post-inoculation, the majority of pigs in both groups had IgG titers of 1:120 or greater (Table 5).

TABLE 5
Serum IgG titers against L. intracellularis from pigs on days −2, 7, 14,
and 20 post-inoculation.

| | | Day | | | |
|---|---|---|---|---|---|
| Groups | Serum titers | −2 | 7 | 14 | 20 |
| Control (n = 4) | Negative | 4 | 4 | 4 | 4 |
| Tissue culture homogenate | Negative | 10 | 10 | 5 | 0 |
| (n = 10) | | | | | |
| | 1:30 | 0 | 0 | 0 | 1 |
| | 1:60 | 0 | 0 | 1 | 3 |
| | 1:120 | 0 | 0 | 3 | 1 |
| | 1:240 | 0 | 0 | 1 | 2 |
| | 1:480 | 0 | 0 | 0 | 3 |
| Intestine homogenate (n = 10) | Negative | 10 | 10 | 7 | 0 |
| | 1:30 | 0 | 0 | 1 | 1 |
| | 1:60 | 0 | 0 | 2 | 0 |
| | 1:120 | 0 | 0 | 0 | 3 |
| | 1:240 | 0 | 0 | 0 | 4 |
| | 1:480 | 0 | 0 | 0 | 1 |
| | 1:960 | 0 | 0 | 0 | 0 |
| | 1:1920 | 0 | 0 | 0 | 1 |

These data demonstrated that the severity of a Lawsonia infection can be assessed by measuring the IgG titers against L. intracellularis in serum samples from pigs. These data also demonstrated that pigs inoculated with a virulent L. intracellularis microorganism develop high titer IgG antibodies against L. intracellularis as the infection persists.

### Example 3

### IgA Titration Assay

The same 96-well plates used for IPMA serologic testing in Example 2 were rehydrated in a solution of PBS containing 5% dry milk for 10 minutes at 37° C. to block nonspecific reactions. The intestinal lavage samples described in Example 1 were thawed and diluted in the same rehydration solution in serial four fold dilutions (1:4, 1:16, and 1:64). Next, 50 μL of each diluted sample was added to a separate well. After incubating for 30 minutes at 37° C., the plates were washed five times with PBST, and 30 μL PBST containing peroxidase-conjugated anti-porcine IgA (1:1000; #A100-102P-11; Bethyl, Montgomery, Tex.) was added to each well. After incubating for 45 minutes at 37° C., the plates were washed five times with PBST, and 100 μL pre-diluted 3-amino-9-ethyl-carbazole chromogen solution (AEC; A-6926; Sigma) was added to each well. After incubating for 20 minutes at room temperature, the plates were washed with PBS three times, allowed to dry, and examined using an inverted light microscope.

All four pigs in the control group were negative for IgA antibodies against L. intracellularis. Two pigs from the tissue culture homogenate group were negative for IgA antibodies, while three pigs, four pigs, and one pig tested positive for IgA antibodies at titers of 1:4, 1:16, and 1:64, respectively. Three pigs from the intestine homogenate group were negative for IgA antibodies, while two pigs, three pigs, and two pigs tested positive for IgA antibodies at titers of 1:4, 1:16, and 1:64, respectively (Table 6). These data demonstrated that an IgA titration assay can be used to identify pigs with a Lawsonia infection.

TABLE 6
Ileum lavage IgA titers against L. intracellularis from pigs on day 22
post-inoculation.

| Groups | Negative | 1:4 | 1:16 | 1:64 |
|---|---|---|---|---|
| Control (n = 4) | 4 | 0 | 0 | 0 |
| Tissue culture homogenate | 2 | 3 | 4 | 1 |
| (n = 10) | | | | |
| Intestine homogenate (n = 10) | 3 | 2 | 3 | 2 |

### Example 4

### Isolating a Lawsonia intracellularis Microorganism

A pig having a L. intracellularis infection was euthanized, and the ileum was surgically removed using sterile forceps and sterile scissors/scalpel. The removed ileum was stored immediately at −70° C.

A 2 cm section was cut from the frozen ileitis mucosa and placed in a sterile petri dish. The mucosal surface was scraped, and 2 mL sucrose-potassium-glutamate (SPG) solution containing 1% trypsin was added to the petri dish. A smear of the mucosal surface was made on a clean glass slide. The smear was air dried and fixed in acetone for 30 seconds. The presence of L. intracellularis in the fixed smear was confirmed by immunofluorescence. Briefly, the fixed smear was stained using a monoclonal antibody specific for L. intracellularis, described elsewhere (McOrist et al., Vet. Rec.,121:421-422, 1987). L. intracellularis microorganisms were visualized using peroxidase-conjugated streptavidin (LSAB2-K0675-11; DAKO USA).

The section was homogenized in the 2 mL SPG/trypsin solution for 30 seconds in a glass tube fitted with a Teflon® pestle. The homogenate was transferred aseptically to a 15 ml conical tube. After agitating at 37° C. for 35 minutes, 10 mL SPG solution containing 10% fetal calf serum (FCS) was added to the homogenate. The resulting mixture was further homogenized in a tissue grinder. Another 10 mL SPG solution containing 10% FCS was added to the homogenate, and the resulting mixture was placed on ice. The mixture was then filtered sequentially through 2.7μ GF/D, 1.0μ and 0.65μ filters. The final filtrate (˜10 mL) was collected in a fresh sterile 15 mL conical tube. The filtrate was aliquoted aseptically into 1 mL cryovials, and the L. intracellularis isolate was designated PHE/MN1-00. The filtrate for immediate infection was held on ice, and the remaining aliquots of the L. intracellularis isolate PHE/MN1-00 were frozen immediately at −70° C. After 24 hours at −70° C., the frozen aliquots of the L. intracellularis isolate PHE/MN1-00 were transferred to liquid nitrogen for long term storage.

A frozen aliquot of the L. intracellularis isolate PHE/MN1-00 was thawed, and the L. intracellularis isolate PHE/MN1-00 propagated in McCoy cells to obtain multiple vials of L. intracellularis isolate PHE/MN1-00. Briefly, the L. intracellularis isolate PHE/MN1-00 was used to infect McCoy cells. After ten passages, the L. intracellularis isolate PHE/MN1-00 was harvested. First, the original supernatant was collected to obtain any extracellular L. intracellularis. Second, the McCoy cells infected with the L. intracellularis isolate PHE/MN1-00 were lysed, and the released L. intracellularis was combined with the extracellular L. intracellularis obtained from the original supernatant. The cell lysis was performed by incubating the infected McCoy cells with 0.1% KCl for five minutes at 37° C. After removing the KCl solution, the McCoy cells were placed in a SPG solution and mechanically disrupted using a glass syringe. The cell debris was removed via low speed centrifugation. The resulting supernatant containing the released L. intracellularis isolate PHE/MN1-00 was combined with the original supernatant containing the extracellular L. intracellularis isolate PHE/MN1-00. Once combined, the solution containing the L. intracellularis isolate PHE/MN1-00 was divided into aliquots that were deposited with the ATCC.

The filtrate for infection was diluted 1:10 in prewarmed (i.e., 37° C.) Dulbecco's modified Eagle medium (DMEM) containing 7% FCS. A 25 cm² tissue culture flask containing a culture of McCoy cells at 30% confluency was inoculated with 1 mL of the diluted filtrate. In addition, two trac bottles (Bibby sterilin-#129AX/1) each containing cultures of McCoy cells at 30% were inoculated with 0.5 mL of the diluted filtrate. The flask and trac bottles were then centrifuged at 4° C. for 30 minutes at 1,000×g). After centrifuging, the caps of the flask and trac bottles were loosened, and the flask and trac bottles were placed in a vacuum jar. The atmosphere in the vacuum jar was evacuated to 500 mm Hg. After evacuating, the vacuum jar was purged with hydrogen gas. The hydrogen gas pressure was then released, and the flask was placed in an incubator under 8.8% CO₂, 8% O₂ and 83.2% N at 37° C. The infected cultures in the flask and trac bottles were refed after 3-6 hours with DMEM containing 7% FCS, 0.5% neomycin, and 1.0% vancomycin. The inoculated cells were passed once every week to fresh cells until 100% of the cells were infected as assessed by immunoperoxidase stain.

Nearly 100% of the inoculated cells were infected 5 weeks after inoculation. These data demonstrate that virulent L. intracellularis microorganisms can be isolated from infected pigs.

### Example 5

### Propagating Lawsonia intracellularis Microorganisms

IEC-18 cells (ATCC CRL 1592) were seeded at 5×10⁵ cells/mL in 5 mL maintenance media consisting of DMEM (11965-092; Gibco BRL) with 7% fetal bovine serum (FBS; F2442; Sigma) and 1% L-glutamine (25030-081; Gibco BRL) in a 25 cm² “donor” flask. The seeded cells formed a confluent monolayer within 72 hours. The confluent monolayer of cells was split every 7 days using standard cell culture techniques. Briefly, the confluent monolayer of cells was washed with 1 mL sterile phosphate buffered saline (PBS) containing 0.02% EDTA. After removing the PBS/EDTA solution completely with a pipette, the cells were trypsinized with 5 mL warm (i.e., 37° C.) trypsin/Versene for 90 seconds at room temperature. The trypsin/Versene solution was decanted, and the cells were further incubated for 10 minutes at 37° C., 5%CO₂ to allow the cells to detach completely from the flask surface. The detached cells were suspended in 5 mL of maintenance media, and a cell count was performed using a Neubauer chamber hemocytometer. The suspended cells were diluted to 7×10⁵ cells/mL in maintenance media, and 0.25 mL aliquots of the diluted cells were added to two new 25 cm² “daughter” flasks containing 5 mL maintenance media. The “donor” flask was reseeded in the same manner as the “daughter” flasks, and all the flasks were incubated for 7 days at 37° C., 5% CO₂.

Prior to infecting cells with L. intracellularis microorganisms, 7×10⁵ cells/mL of diluted cells were seeded into a 175 cm² flask containing 35 mL of maintenance media. The seeded cells were grown for 24 hours (i.e., 10-20% confluent). After confirming that the cells were 10-20% confluent, a 1 mL aliquot of SPG media containing 10⁷ L. intracellularis microorganisms/mL was added to the flask. The cap of the flask was loosened, and the flask was placed in an anaerobic bacteriologic jar. The atmosphere in the bacteriologic jar was evacuated to 500 mm Hg. After evacuating, the bacteriologic jar was purged with hydrogen gas. The hydrogen gas pressure was then released, and the flask was placed in an incubator under 8.8% CO₂, 8% O₂ and 83.2% N at 37° C. After 5 to 7 days, the following culture characteristics were evaluated visually: media color, monolayer confluency, cytoplasmic vacuolization, and quantity of bacteria in media. These culture characteristics were reevaluated every two days. At 2 and 4 days after infecting the cells, the media was removed, and 10-15 mL infection media consisting of DMEM with 5% FBS, 1% L-glutamine, and 0.5% amphotericin B (30-003-CI; Mediatech Inc., Herndon, Va.) was added to the cells. The infection media can also contain a combination of 1% vancomycin (V2002; Sigma) and 0.5% neomycin (N1012; Sigma).

Six days after infecting cells, the infection was passed by splitting the infected cells 1:3 into 175 cm² flasks containing cells having been grown for 24 hours (i.e., 10-20% confluent). Briefly, the infected cell monolayer was removed from the flask surface using a cell scraper, and the resulting cell suspension was transferred to a 50 mL conical tube. The cell suspension was centrifuged for 20 minutes at 5,000 RPM in a Beckman TA-10 rotor producing a cell pellet. The supernatant was discarded, and the cell pellet was resuspended in 3 mL of fresh infection media. One mL of the resuspended cell pellet was added to each 175 cm² flask containing cells having been grown for 24 hours (i.e., 10-20% confluent). The flasks were evacuated, purged, and maintained under the same conditions described above. Typically, the amount cells containing bacteria obtained from 27 175 cm² flasks of infected cells was sufficient to challenge 12 five-week old pigs.

### Example 6

### Preparing Lawsonia intracellularis Microorganisms for Vaccine Challenges

The L. intracellularis inoculum for challenging pigs was prepared using the infected cells described in Example 4. Briefly, the infected cell monolayers from 27 175 cm² flasks were removed from the surfaces of the flasks using a cell scraper. The resulting cell suspensions were transferred to 50 mL conical tubes. The cell suspension was centrifuged for 20 minutes at 5,000 RPM in a Beckman TA-10 rotor producing a cell pellet. The supernatants were discarded, and the cell pellets were resuspended in 3 mL sucrose-potassium-glutamate (SPG) solution containing 0.218M sucrose, 0.0038M KH₂PO₄, 0.0072M K₂HPO₄, 0.0049M potassium glutamate, and 5% FBS. The resuspended cell pellets were pooled in a glass bottle. The final volume of inoculum was between 350 and 400 mL of SPG containing 108 L. intracellularis microorganisms per mL.

### Example 7

### Testing Lawsonia intracellularis Viability

IEC-18 cells (ATCC CRL 1592) were seeded at 5×10⁵ cells/mL in 10 mL maintenance media consisting of DMEM (11965-092; Gibco BRL) with 7% fetal bovine serum (FBS; F2442; Sigma) and 1% L-glutamine (25030-081; Gibco BRL) in a two-well chamber slide. The seeded cells were grown to 10-20% confluency. After confirming that the monolayer was 10-20% confluent, a cryogenic vial containing 1 mL 10⁷ L. intracellularis microorganisms per mL was thawed. The 1 mL aliquot was diluted with 9 mL maintenance media. After removing the maintenance media from the two-well chamber slide, a 1 mL aliquot of the diluted L. intracellularis microorganisms was added to each of the chambers in the chamber slide. The chamber slide was placed in an anaerobic bacteriologic jar, and the atmosphere in the jar was evacuated to 500 mm Hg. After evacuating, the jar was purged with a gas containing 10% hydrogen, 10% carbon dioxide, and 80% nitrogen (i.e., 10-10-80 gas). The gas was released, and again the atmosphere in the jar was evacuated to 500 mm Hg. The jar was again purged with 10-10-80 gas, and then placed at 37° C., 5% CO₂. The following culture characteristics were evaluated visually every 48 hours: media color, monolayer confluency, cytoplasmic vacuolization, and quantity of bacteria in media. At 2 and 4 days after infecting the cells, the media was removed, and 1 mL infection media consisting of DMEM with 5% FBS, 1% L-glutamine, and 0.5% amphotericin B (30-003-CI; Mediatech Inc.) was added to each of the chambers in the chamber slide. The infection media can also contain a combination of 1% vancomycin (V2002; Sigma) and 0.5% neomycin (N1012; Sigma). The process of evacuating and purging the jar was repeated every 48 hours.

Seven days after infecting the cells in the chamber slide, the infection media was discarded and the cells in each of the chambers were fixed in cold acetone for 30 seconds. After removing the cold acetone and allowing the chamber slide to dry, the fixed cells were rehydrated with PBS for 10 minutes at room temperature. The PBS was removed, and 200 μL of a DMEM solution containing monoclonal antibodies against L. intracellularis (McOrist et al, 1987, Vet. Rec., 121: 421-422) was added to each chamber in the chamber slide. After incubating the chamber slide for 30 minutes at 37° C., the cells were washed with PBS, and 200 μL of IPX solution (PBS with 0.08% Tween-80 and 0.1% bovine serum albumin (BSA)) containing peroxidase-conjugated anti-mouse IgG (1:25; A 5906; Sigma) was added to each chamber in the chamber slide. After incubating the chamber slide for 45 minutes at 37° C., the cells were washed with PBS. The washed cells were then treated with 200 μL of a 3,3′ diaminobenzidine tetrahydrochloride/H₂O₂ (DAB) solution. After incubating for 5 minutes at room temperature, the treated cells were washed with PBS containing Harris hematoxylin for 40 seconds at room temperature. The cells were then further washed with distilled water and allowed to dry. The coverslips containing the dried cells were mounted using a mounting media, and the cells were observed by light microscopy.

Several DAB-positive foci containing more than 30 bacteria were seen in the cytoplasm of the cells, indicating that the L. intracellularis microorganisms in the vial were viable and capable of infecting cells.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated Lawsonia intracellularis microorganism, wherein administration of said microorganism to a pig produces at least one gross lesion.

2. The isolated Lawsonia intracellularis microorganism of claim 1, wherein administration of said microorganism to said pig produces a gross lesion to the same degree as the gross lesion produced when the microorganism of ATCC accession number ______ (L. intracellularis isolate PHE/MN1-00) is administered to a susceptible pig.

3. The isolated Lawsonia intracellularis microorganism of claim 1, wherein said microorganism has the identifying characteristics of the microorganism of ATCC accession number ______ (L. intracellularis isolate PHE/MN1-00).

4. The isolated Lawsonia intracellularis microorganism of claim 1, wherein said microorganism was obtained from the deposit having ATCC accession number ______ (L. intracellularis isolate PHE/MN1-00).

5. The isolated Lawsonia intracellularis microorganism of claim 1, wherein said microorganism was frozen.

6. The isolated Lawsonia intracellularis microorganism of claim 1, wherein said microorganism was cultured for more than one passage.

7. The isolated Lawsonia intracellularis microorganism of claim 1, wherein said microorganism was cultured for more than two passages.

8. The isolated Lawsonia intracellularis microorganism of claim 1, wherein said microorganism was cultured for more than five passages.

9. The isolated Lawsonia intracellularis microorganism of claim 1, wherein said microorganism was cultured for more than ten passages.

10. The isolated Lawsonia intracellularis microorganism of claim 1, wherein said microorganism was cultured for less than twenty passages.

11. The isolated Lawsonia intracellularis microorganism of claim 1, wherein said microorganism was cultured for less than ten passages.

12. The isolated Lawsonia intracellularis microorganism of claim 1, wherein said microorganism was cultured for less than five passages.

13. The isolated Lawsonia intracellularis microorganism of claim 1, wherein said microorganism is in media.

14. The isolated Lawsonia intracellularis microorganism of claim 1, wherein said microorganism is in a eukaryotic cell.

15. The isolated Lawsonia intracellularis microorganism of claim 14, wherein said eukaryotic cell is selected from the group consisting of McCoy, INT407, and IEC-18 cells.

16. A method for determining the efficacy of a PE treatment material, said method comprising:
a) obtaining a vertebrate treated with said material, infecting said vertebrate with a Lawsonia intracellularis microorganism, wherein administration of said Lawsonia intracellularis microorganism to a susceptible pig produces at least one gross lesion, and
b) determining the presence, absence, or severity of a Lawsonia infection in said vertebrate, wherein the degree of said Lawsonia infection inversely reflects the level of protection provided by said material.

17. The method of claim 16, wherein said material is a vaccine.

18. The method of claim 17, wherein said vaccine is selected from the group consisting of attenuated live vaccines, modified live vaccines, recombinant vaccines, subunit vaccines, and nucleic acid vaccines.

19. The method of claim 16, wherein said material is selected from the group consisting of an antibiotic, a feed additive, and an antimicrobial agent.

20. The method of claim 16, wherein said material lacks live Lawsonia microorganisms.

21. The method of claim 16, wherein said infecting comprises a delivery method selected from the group consisting of intranasal administration, oral administration, intragastric administration, subcutaneous administration, and intramuscular administration.

22. The method of claim 16, wherein said vertebrate is selected from the group consisting of pigs, horses, hamsters, mice, rats, rabbits, and chickens.

23. The method of claim 16, wherein administration of said microorganism to said susceptible pig produces a gross lesion to the same degree as the gross lesion produced when the microorganism of ATCC accession number (L. intracellularis isolate PHE/MN1-00) is administered to a different susceptible pig.

24. The method of claim 16, wherein said microorganism has the identifying characteristics of the microorganism of ATCC accession number ______ (L. intracellularis isolate PHE/MN1-00).

25. The method of claim 16, wherein said microorganism was obtained from the deposit having ATCC accession number ______ (L. intracellularis isolate PHE/MN1-00).
